(19) United States
(12) Patent Application Publication
(10) Pub. No.: US 2003/0032183 A1
(43) Pub. Date: Feb. 13, 2003

(54) Stem cell differentiation

(76) Inventor: Steven D. Sheridan, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/156,602

(22) Filed: May 23, 2002

Related U.S. Application Data
(60) Provisional application No. 60/293,582, filed on May 25, 2001.

(51) Int. Cl. C12N 5/08

(52) U.S. Cl. 435/370

(57) ABSTRACT

Treatment of stem cells with a retinoid induces differentiation of the stem cells into hepaticopancreatic tissue.

### RELATED APPLICATION INFORMATION

This application claims priority to U.S. Provisional Patent Application No. 60/293,582, filed May 25, 2001, which is hereby incorporated by reference in its entirety.

### BACKGROUND OF THE INVENTION

1. Field of the Invention

This invention relates to methods of inducing stem cell differentiation, and particularly to methods of inducing stem cells to form hepaticopancreatic tissue by treating the stem cells with retinoids.

2. Description of the Related Art

Hepaticopancreatic disorders and extraintestinal gastrointestinal disorders affect millions of people around the world. Examples of such disorders include diabetes, pancreatitis, hepatic cirrhosis, hepatitis, cancer and pancreatico-biliary disease. Existing treatments for these disorders are only partially satisfactory. For example, diabetes is divided into two types depending on the age of onset and the mechanism by which the body loses control over blood glucose levels. Type I diabetes (juvenile diabetes) is characterized by an auto-immune destruction of the insulin-producing beta-cells contained in the islets of Langerhans of the pancreas and is usually seen in younger patients. This type has been treated by ectopic injections of purified insulin at prescribed times as dictated by measurements of the blood sugar levels. Though this treatment is beneficial, long-term effects of transient abnormal glucose levels leads to a gradual destruction of other organs resulting in kidney failure, limb amputation and blindness. Type II diabetes generally occurs in older patients and is characterized by an inability to respond to the production of insulin (insulin-independent) leading ultimately to diabetes and a subsequent loss of pancreatic beta cells.

Recently, the ability to transplant isolated beta-cell containing pancreatic islets has been demonstrated to have the potential of eliminating the need for insulin injection and to resume normal blood glucose regulation. The technical difficulty in this procedure, however, arises from the lack of suitable organs from which to isolate these structures and the intrinsic instability of the pancreas once removed from donors. Thus, the efficacy of transplantation is limited by the unavailability of large enough amounts of endocrine insulin-producing cells (IPCs).

N. Moriya et al. have reported the formation of pancreas-like structures from the treatment of presumptive ectoderm tissue with activin and retinoic acid, see “In Vitro Pancreas Formation From Xenopus Ectoderm Treated with Activin and Retinoic Acid,” Develop. Growth Differ., Vol. 42, pp. 593-602 (2000). D. Stafford and V. Prince have recently reported that in Zebrafish development the formation of all pancreatic cell types is dependent on retinoid signaling, see “Pancreatic Development, Proliferation and Stem Cells,” meeting abstract, Oct. 18-19, 2001 National Institutes of Health. R. McKay et al. have reported the differentiation of embryonic stem cells to insulin-secreting structures by plating embryoid bodies into a serum-free medium, see “Differentiation of Embryonic Stem Cells to Insulin-Secreting Structures Similar to Pancreatic Islets,” Science Vol. 292, pp 1389-1394 (2001).

### SUMMARY OF THE INVENTION

It has now been discovered that the use of retinoids causes stem cells to differentiate into hepaticopancreatic tissue lineages such as pancreatic tissue and liver tissue. Using the methods described herein, hepaticopancreatic tissue can be produced in the laboratory and people or animals suffering from hepaticopancreatic disorders or extraintestinal gastrointestinal disorders can then be treated by transplantation of these hepaticopancreatic tissues.

In a preferred embodiment, a method of inducing stem cell differentiation is provided, comprising treating isolated stem cells with a retinoid under conditions effective to cause at least a portion of the stem cells to differentiate into hepaticopancreatic tissue. Preferably, the retinoid is retinoic acid and the hepaticopancreatic tissue is pancreatic endocrine tissue.

In another preferred embodiment, a composition comprising hepaticopancreatic tissue is provided, wherein the composition is produced by a method comprising treating isolated stem cells with a retinoid under conditions effective to cause at least a portion of the stem cells to differentiate into hepaticopancreatic tissue. Preferably, the composition comprises pancreatic endocrine tissue.

In another preferred embodiment, a method of treatment is provided, comprising identifying a mammal having an extraintestinal gastrointestinal disorder and administering to the mammal a therapeutically effective amount of a composition, wherein the composition is produced by a method comprising treating isolated stem cells with a retinoid under conditions effective to cause at least a portion of the stem cells to differentiate into hepaticopancreatic tissue. Preferably, the extraintestinal gastrointestinal disorder is a hepaticopancreatic disorder and the mammal is a human.

### BRIEF DESCRIPTION OF THE DRAWINGS

FIG. 1 shows photographs of gel electrophoresis results obtained as a result of RT-PCR analyses on embryonic stem cells differentiated in the presence of retinoic acid, as compared to embryonic stem cells differentiated in the absence of retinoic acid.

FIG. 2 is a plot showing the blood glucose levels of mice either sham treated or treated with differentiated ES cells as a function of time.

FIG. 3 shows photomicrographs of transplanted tissue sections stained with anti-insulin antibodies.

FIG. 4 shows photomicrographs of embryonic stem cells differentiated in the presence of retinoic acid. Panels indicate negative control lacking primary antibody (FIG. 4A) or insulin specific staining after the addition of primary antibody (FIG. 4B).

FIG. 5 is a plot illustrating the effect of differentiating stem cells in the presence of various morphogen/retinoic acid combinations, as determined by measuring the insulin content of the resulting differentiated stem cells.

### DETAILED DESCRIPTION OF THE PREFERRED EMBODIMENT

A preferred embodiment involves inducing cell differentiation by treating stem cells with a retinoid. “Stem cells” are self-renewing cells that can generate the many cell types in the body. Stem cells may be obtained from various sources by methods known to those skilled in the art. Preferred stem cells are isolated stem cells, preferably isolated from a stem cell source selected from the group consisting of placenta, bone marrow, blood, adipose tissue, neural tissue, umbilical cord blood, blastocyst inner cell mass, and germ cells. Most preferably, isolated stem cells are mammalian embryonic stem cells. “Isolated” stem cells contain a higher weight fraction of stem cells than the source from which they are obtained.

The stem cell differentiation methods described herein are preferably practiced on relatively large numbers of stem cells in order to produce clinically useful amounts of differentiated stem cells. Various methods are known in the art for producing such large amounts of stem cells. For example, stem cells may be cultured by various known techniques to encourage growth and proliferation, see E. J. Robertson “Teratocarcinomas and Embryonic Stem Cells: A Practical Approach”, IRL Press (1987). Isolated stem cells may be in the form of embryoid bodies, such as those produced by culturing stem cells.

Stem cells, preferably isolated stem cells, are preferably treated with a retinoid to cause at least a portion of the stem cells to differentiate into hepaticopancreatic tissue. A “retinoid” is a member of the class of compounds consisting of four isoprenoid units joined in a head-to-tail manner, see G. P. Moss, “Biochemical Nomenclature and Related Documents,” 2^{nd} Ed. Portland Press, pp. 247-251 (1992). “Vitamin A” is the generic descriptor for retinoids exhibiting qualitatively the biological activity of retinol. Preferred retinoids are molecules represented by the formula (I), wherein the double bonds can each individually be cis or trans and wherein R is selected from the group consisting of CH₂OH, CHO, CO₂H, CH₃, CH₂OCOCH₃, CH₂NH₂, CH═NOH, CH═N(CH₂)₄CHNH₂CO₂H, CO₂C₂H₅, and beta-D-glucopyranuronic acid.

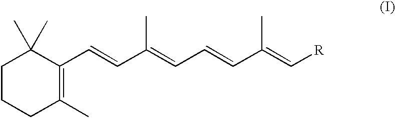

Other preferred retinoids include seco retinoids, in which the ring of formula (I) is opened up with the addition of one or more hydrogen atoms at each terminal group thus created; nor_retinoids, in which a CH₃, CH₂, CH or C group has been eliminated from a retinoid; and retro retinoids, in which the conjugated polyene system has been shifted by one position. Highly preferred retinoids are retinoic acid (R=CO₂H), retinol (R=CH₂OH) and retinal (R=CHO).

The term “hepaticopancreatic tissue” means liver tissue or pancreatic tissue, including pancreatic endocrine tissue, pancreatic exocrine tissue, and insulin-producing cells. Stem cells are preferably treated with a retinoid under conditions effective to cause at least a portion of the stem cells to differentiate into hepaticopancreatic tissue. Preferred conditions include contacting isolated stem cells with a retinoid at a temperature in the range of about 0° C. to about 45° C., preferably about 37° C., and varying the time/retinoid concentration conditions to favor differentiation. The retinoid is preferably provided in the form of an aqueous solution so that the concentration of the retinoid can be accurately controlled.

Contacting the cells with the retinoid can be brief, e.g. a few seconds, in which case the retinoid concentration is preferably relatively high, e.g. about 1 molar (1 M) or less, or contacting with retinoid can be rather prolonged, e.g., weeks, in which case the retinoid concentration is preferably relatively low, e.g., about 1 micromolar (1 μM) or greater. Thus, retinoid concentration during contacting can vary over a broad range, preferably about 1 μM or greater, more preferably about 100 μM or greater, preferably about 1 M or less, more preferably about 0.01 M or less. Likewise, the time for contacting can also vary over a broad range, preferably about 10 seconds or greater, more preferably about 1 hour or greater, preferably about 2 weeks or less, more preferably about 4 days or less. “Contacting” is used in a broad sense to include all manner of different ways of contacting the stem cells with the retinoid, whether actively agitated or not. Thus, contacting includes but is not limited to washing the stem cells in a retinoid solution, suspending the stem cells in a retinoid solution, gently stirring the stem cells in a retinoid solution, adding a retinoid solution to a monolayer of stems cells on a substrate, etc. Preferably, stem cells are contacted with a retinoid using a short initial period of gentle agitation followed by a period of relative quiescence. In another delivery embodiment, retinoid molecules can also be attached to other solid/peptide/protein or small molecule support structures (e.g., matrix molecules, other drugs/peptides, or solid surfaces such as culture dishes, beads, or substrate attachment factors).

In a preferred method, stem cells are treated with a retinoid under conditions effective to cause at least a portion of the stem cells to differentiate into pancreatic tissue. Preferably, the pancreatic tissue comprises pancreatic endocrine tissue, more preferably insulin-producing cells. Most preferably, the insulin-producing cells are glucose-responsive. “Glucose-responsive” means that the insulin output of the cells changes in response to the glucose level. In another preferred embodiment, the hepaticopancreatic tissue comprises liver tissue.

In a preferred embodiment, the stem cells are contacted with a retinoid and a morphogen. In this context, a “morphogen” is a synthetic or natural compound or protein factor which induces the differentiation of cells. Examples of preferred morphogens include members of the glucagon-like peptide family (e.g. GLP-1, exendin-4, etc., see T. J. Kieffer and J. F. Habener, “The Glucagon-Like Peptides,” Endocrinology Reviews Dec 1999, Vol 20, no. 6 pp 876-913), cAMP raising agents (e.g., forskolin, IBMX, thephyline and the like), nicotinamide, acetycholine and related molecules, transcription factors (e.g., PDX-1, Ngn-3, etc., see M. Sander and M. S. German “The beta cell transcription factors and development of the pancreas,” Journal of Molecular Medicine May 1997, Vol 75, no. 5, pp 327-40), protein growth factors (e.g., gastrin, gastrin-releasing peptide, hepatocyte growth factor, betacellulin, etc., see H. Edlund, “Factors controlling pancreatic cell differentiation and function,” Diabetologia September 2001, Vol 44, no. 9, pp 1071-9), and mixtures thereof. Each of the aforementioned articles is incorporated by reference in its entirety, and especially for the purpose of describing morphogens. More preferably, the morphogen is exendin-4, gastrin, and/or gastrin releasing peptide and mixtures thereof.

The morphogen may be contacted with the stem cells in the general manner described herein for contacting stem cells with a retinoid. The stem cells may be contacted with the retinoid and morphogen in any order or simultaneously. Preferably, the stem cells are contacted with a retinoid during an initial stage, then with a morphogen or a combination of morphogen and retinoid during a later stage to further differentiate the stem cells. In preferred embodiments, the combination of retinoid and morphogen produces greater amounts of differentiated hepaticopancreatic tissue than the use of either agent alone.

A preferred embodiment provides compositions comprised of differentiated stem cells or hepaticopancreatic tissue produced by any of the methods described herein. As produced, such compositions preferably comprise about 1% or more of hepaticopancreatic tissue, more preferably about 10% or more, most preferably about 50% or more, by weight based on total weight of the composition. In a preferred embodiment, such compositions result from conditions that are effective to differentiate at least about 1% of the stem cells into hepaticopancreatic tissue, more preferably about 5% or more, most preferably about 25% or more, by weight based on total weight of the stem cells. Amounts of differentiated stem cells or hepaticopancreatic tissue can be determined by various methods, preferably by gene expression analysis (e.g. RT-PCR), protein expression (e.g., western blotting or immuno-based assays), insulin radio-immuno or ELISA assays, and/or fluorescence activated cell sorting (FACS) with tissue/cell-specific markers. A combination of gene expression analysis, protein expression and FACS is preferably used to determine the amount of islet/beta cells.

Compositions comprising differentiated stem cells or hepaticopancreatic tissue as described herein can comprise other components such as water, stabilizers, salts, opaque tracing materials, heparin, proteins, polypeptides, etc.

Preferred compositions can also be produced by purifying compositions comprising hepaticopancreatic tissue to increase the level of hepaticopancreatic tissue contained therein and/or to reduce the levels of other tissues that may also be produced. Various methods may be used to purify compositions comprising hepaticopancreatic tissue. Preferred methods include transgenic methods and physical methods. Various transgenic methods are known in the art, see e.g., U.S. Pat. No. 6,015,671, which is hereby incorporated by reference in its entirety and especially for the purpose of describing transgenic methodology. Transgenic methods generally involve genetic modification of either the hepaticopancreatic tissue or the other tissue to increase or decrease vulnerability to a specified condition. For example, transgenic manipulation of the stem cells can be used to render the hepaticopancreatic tissue specifically resistant to certain drug treatments, where the other tissue is preferably sensitive to these same treatments. The hepaticopancreatic tissue is preferably recovered in a purified form by collecting the surviving tissue after drug treatment. In addition, physical purification methods can be performed which include known techniques such as staining and sorting by hand and automated methods such as FACS (Fluorescence Activated Cell Sorting) or affinity purification, e.g., affinity chromatography, magnetic bead purification, immunoprecipitation, etc.

Larger amounts of hepaticopancreatic tissue can be produced by genetically engineering a conditionally immortal cell line of hepaticopancreatic tissue to grow indefinitely under laboratory conditions at, e.g., 30° C., but then to grow normally when implanted into the body at 37° C. Methods of creating such immortal cell lines are known, see M. J. O'Hare et al. “Conditional Immortalization of Freshly Isolated Human Mammary Fibroblast ands Endothelial Cells,” Proc. Nat. Acad. Sci., Vol. 98, pp. 646-651 (2001).

A preferred embodiment provides methods of treatment comprising identifying a mammal having a extraintestinal gastrointestinal disorder and administering to the mammal a therapeutically effective amount of a composition comprised of hepaticopancreatic tissue as described herein. An “extraintestinal gastrointestinal” disorder is a disorder of the gastrointestinal tract that is primarily localized in an area other than the interior of the intestine. Non-limiting examples of extraintestinal gastrointestinal disorders include hepaticopancreatic disorders, duodenum disorders, bile duct disorders, appendix disorders, spleen disorders, and stomach disorders. “Hepaticopancreatic” disorders are disorders of the pancreas and liver. Non-limiting examples of hepaticopancreatic disorders include diabetes, pancreatitis, hepatic cirrhosis, hepatitis, cancer and pancreatico-biliary disease. Humans are preferred mammals for treatment purposes. A “disorder” of a particular organ or structure includes situations where the organ or structure is entirely absent. For example, for the purposes of this invention, a person who lacks a pancreas has a pancreas disorder.

Compositions comprised of hepaticopancreatic tissue can be administered to subjects in a variety of ways. Preferably, the compositions are injected directly into a target organ. For example, a composition comprised of pancreatic endocrine tissue can be injected into the pancreas, a composition comprised of liver tissue can be injected into the liver, etc. Compositions comprised of one kind of tissue can be injected into organs comprised of a different type of tissue. For example, pancreatic tissue can be injected into the liver. Methods of implanting exogenous tissue are well known, see, e.g., J. Shapiro, et. al., “Islet Transplantation in Seven Patients With Type 1 Diabetes Mellitus Using Glucocorticoid-free Immunosuppressive Regimen”, New Eng. Jour. Med. Vol. 343, pp 230-238.

In another embodiment of the invention, hepaticopancreatic cells or tissues formed from differentiated stem cells may be encapsulated into, e.g., devices or microcapsules. In one example, the hepatic or pancreatic cells resulting from the differentiation process may be contained in a device which is viably maintained outside the body as an extracorporeal device. Preferably, the device is connected to the blood circulation system such that the differentiated cells can be functionally maintained outside of the body and serve to assist liver or pancreas failure conditions. In a second example, the encapsulated cells may be placed within a specific body compartment such that they remain functional for extended periods of time in the absence or presence of immunosuppressive or immuno-modulatory drugs.

Compositions comprised of hepaticopancreatic tissue are preferably administered to subjects in a therapeutically effective amount. For humans, such amounts are generally determined from the results of clinical trials conducted in accordance with well established protocols. For animals, routine experimentation can be used to establish therapeutically effective amounts for a particular disorder and a particular composition.

It will be appreciated by those skilled in the art that various omissions, additions and modifications may be made to the methods and compositions described herein without departing from the scope of the invention, and all such modifications and changes are intended to fall within the scope of the invention as defined by the claims below.

### EXAMPLES 1-10

Embryonic stem cell lines were cultured and spilt 1:8 every three days for 4 passages on gelatin coated Tissue Culture (TC) dishes without Mouse Embryonic Fibroblasts (MEF's) (with 1500 units/ml Lymphocyte Inhibitory Factor (LIF) in media) to remove MEF's from culture. The resulting stem cells were then differentiated as follows:

On day 1, the stem cells were treated with trypsin to break up some aggregation and then suspended in 1% Fetal Calf Serum (FCS) Media (without LIF). The stem cells were then allowed to self-aggregate into embryoid bodies in suspension culture in bacterial petri dishes. On day 3, the cells were given a fresh media change and then split among two bacterial petri dishes (sample and control). A solution containing 1 μM retinoic acid was intermixed with the sample and both the control (no retinoic acid) and the sample were allowed to sit at 37° C. Fresh media were supplied at day 5 (with fresh 1 μM retinoic acid for the treated sample). At day 7, fresh media was supplied for both, with no retinoic acid (retinoic acid only present from days 3 to 7).

Fresh media was supplied again on day 9. On day 1 the cells were again trypsinized and then placed into TC dishes with 10% FCS media (no LIF). Small aliquots were taken at various times (days 14, 17, 19, 22, and 25) from the cultures and were saved for later analysis by reverse transcriptase polymerase chain reaction (RT-PCR).

On day 14, the media was changed for the two groups of cells (10% FCS) in each population (control and sample). On day 17, the media was changed again.

On day 19, adherent cells were gently blown off by pipetting, then trypsinized and resuspended in 10% FCS in bacterial petri dish suspension cultures. On days 22 and 25, the remaining cells were collected and a portion retained for RT-PCR analysis.

All culturing from day 1 forward was performed in 25 millimolar (mM) glucose (high glucose) until after day 19, when it was changed to 5.5 millimolar glucose (lower glucose) The glucose concentrations ranged from 30 mM to 10 mM on the high end and 0.5 mM-5 mM on the low end.

Total RNA from each aliquot collected above was purified as instructed with a Qiagen RNeasy® Mini purification kit (obtained commercially from Qiagen Inc.). The presence of specific RNA transcripts (i.e. insulin) was determined by RT-PCR using gene specific oligonucleotide primers as instructed with a Qiagen® OneStep RT-PCR kit (obtained commercially from Qiagen Inc.). Total RNA was prepared from cultures of differentiating ES cells. RT-PCR analyses were performed with appropriate oligonucleotide primers (INS-insulin) or the pancreatic specific product amylase (AML).

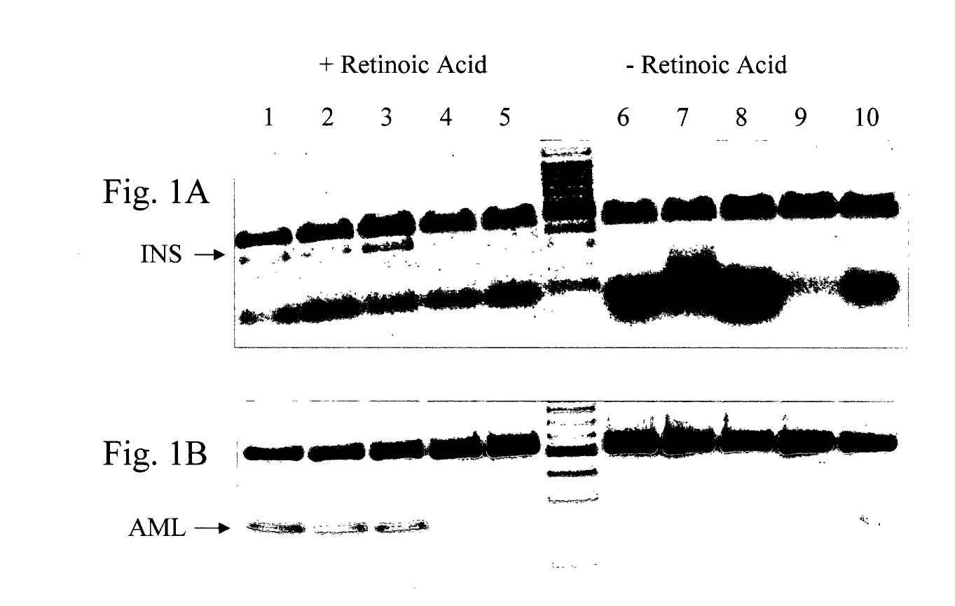

The RT-PCR results summarized in Table 1 show that no insulin was produced in any of the control samples, indicating an absence of insulin or amylase producing cells. In contrast, insulin-producing cells resulted when stem cells were treated with retinoic acid, as indicated by the presence of a correctly sized band during gel electrophoresis of insulin-specific RT-PCR generated products of RNA purified from aliquots obtained at days 14, 17, 19 and 22 (see FIG. 1). Lanes 1-5 and 6-10 in FIG. 1 correspond to time points (see Table 1) taken during the process with or without retinoic acid treatment, respectively. The intensity of the band corresponds to the abundance of RT-PCR product.

TABLE 1

| | | | | Abundance of | Abundance of |
|---|---|---|---|---|---|
| | | Glucose | | Insulin | Amylase |
| Ex. | Lane number | level | | RT-PCR | RT-PCR |
| No. | (see FIG. 1) | (mM) | Day | Product (INS) | Product (AML) |
| 1C | 6 | 25 | 14 | − | − |
| 2C | 7 | 25 | 17 | − | − |
| 3C | 8 | 25 | 19 | − | − |
| 4C | 9 | 5.5 | 22 | − | − |
| 5C | 10 | 5.5 | 25 | − | − |
| 6 | 1 | 25 | 14 | +++ | +++ |
| 7 | 2 | 25 | 17 | +++ | +++ |
| 8 | 3 | 25 | 19 | +++ | +++ |
| 9 | 4 | 5.5 | 22 | + | +/− |
| 10 | 5 | 5.5 | 25 | − | − |

C: Control
+: Indicates relative abundance
−: No RT-PCR band observed

### EXAMPLES 11-12

The differentiated ES cells described above in Examples 1-10 were cultured for an additional 7 days to day 32. At this point the differentiated cell clusters were stained with the vital dye dithizone (DTZ). DTZ is a specific dye for zinc-containing granules that are especially abundant in differentiated beta cells and are representative of insulin-containing storage structures (see Z. A. Latif, J. Noel, and R. Alejandro, “A simple method of staining fresh and cultured islets.” Transplantation, 1988. Vol. 45, no. 4: pp 827-30). Approximately 200-300 DTZ positively stained cell clusters were transplanted under the kidney capsule of streptozotocin (STZ) induced diabetic severe combined immuno-deficient (SCID) mice to evaluate their ability to reverse the diabetic state of the animal (see Wilson, G. L. and E. H. Leiter, “Streptozotocin interactions with pancreatic beta cells and the induction of insulin-dependent diabetes,” Current Topics Microbiol. Immunol. 1990, Vol. 156 pp 27-54).

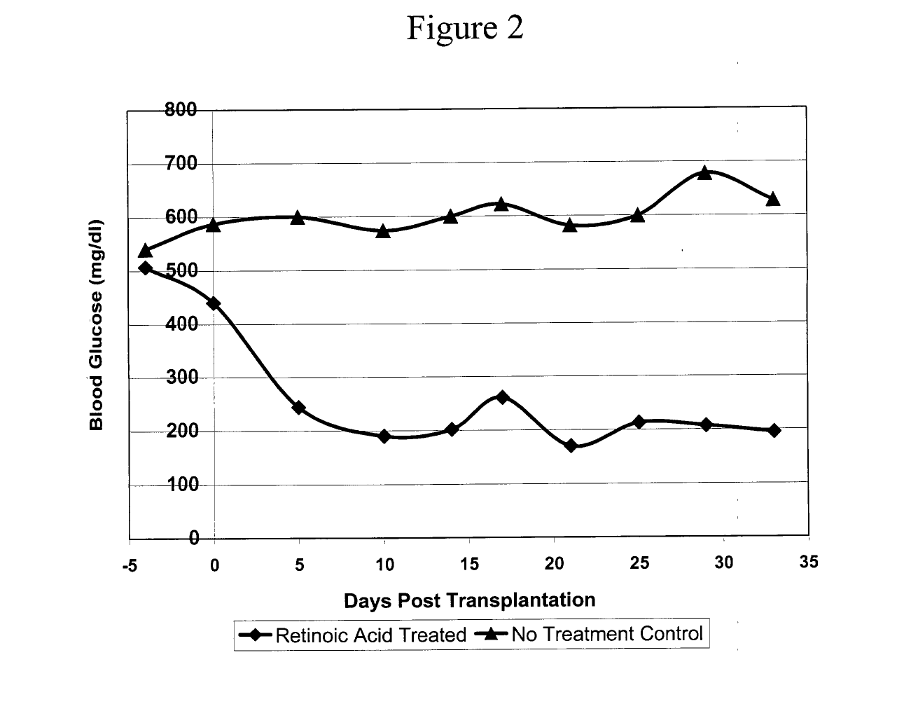

The graph in FIG. 2 illustrates the ability of retinoic acid-treated differentiated embryonic stem cells to correct the blood glucose levels in STZ-SCID mice after transplantation. FIG. 2 also shows that the blood glucose levels of sham treated control mice (operated on, but not transplanted with retinoic acid-treated differentiated embryonic stem cells) were not corrected.

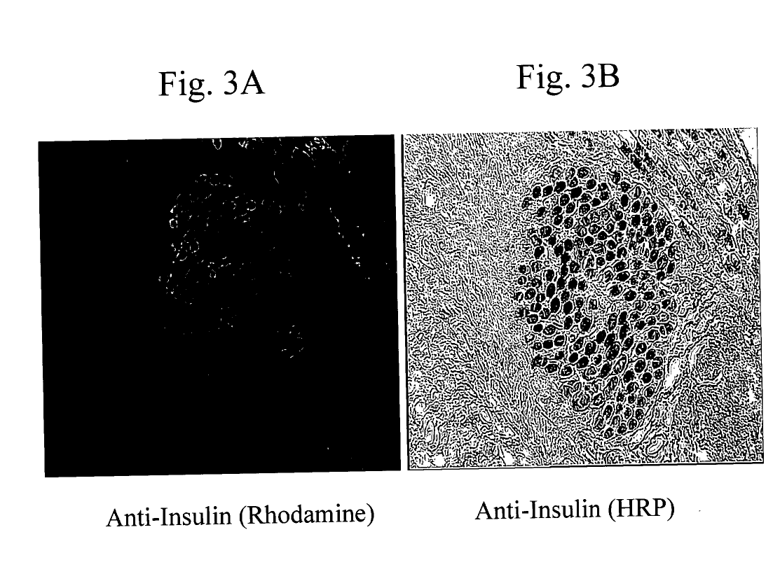

The transplanted tissue was removed, fixed with formalin, embedded in paraffin blocks then sectioned for either fluorescent (rhodamine) or peroxidase (HRP) immunohistochemical analysis. The photomicrograph shown in FIG. 3 demonstrates the presence of insulin protein in the transplanted retinoic acid-treated differentiated tissue as determined by specific antibody staining.

### EXAMPLES 13-14

Embryonic stem cell lines were cultured as described above for Examples 1-10 on gelatin coated Tissue Culture (TC) dishes without Mouse Embryonic Fibroblasts (MEF's) (with 1500 units/ml Lymphocyte Inhibitory Factor (LIF) in media) to remove MEF's from culture. The resulting stem cells were then differentiated as described above (with retinoic acid during treatment during days 3 to 7) except that the formed embryoid bodies were maintained in suspension for the duration of the experiment as opposed to being separated and adhered to TC dishes. All culturing from day 1 forward was performed in 25 millimolar (mM) glucose (high glucose) until after day 19, when it was changed to 5.5 millimolar glucose (physiological glucose).

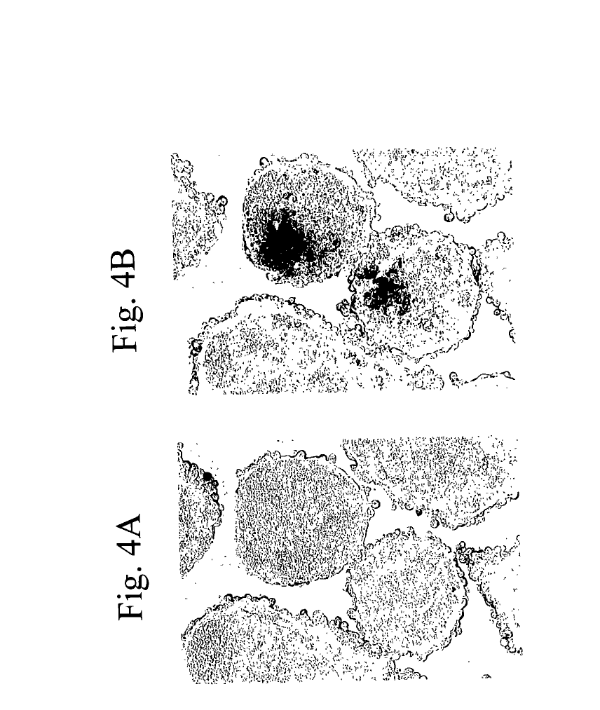

On day 32, suspended embryoid bodies were collected, fixed with formalin, embedded in paraffin blocks, then sectioned for immunohistochemical analysis. immunoperoxidase cytochemistry was used to localize insulin in differentiated cellular aggregates treated with retinoic acid. The photomicrographs reproduced in FIG. 4 demonstrate the presence of insulin protein in a number of the retinoic acid treated embryoid bodies as determined by specific antibody staining (FIG. 4B) as compared to a control sample lacking the primary antibody (FIG. 4A). These results show that the embryoid bodies treated with retinoic acid produce insulin.

### EXAMPLES 15-21

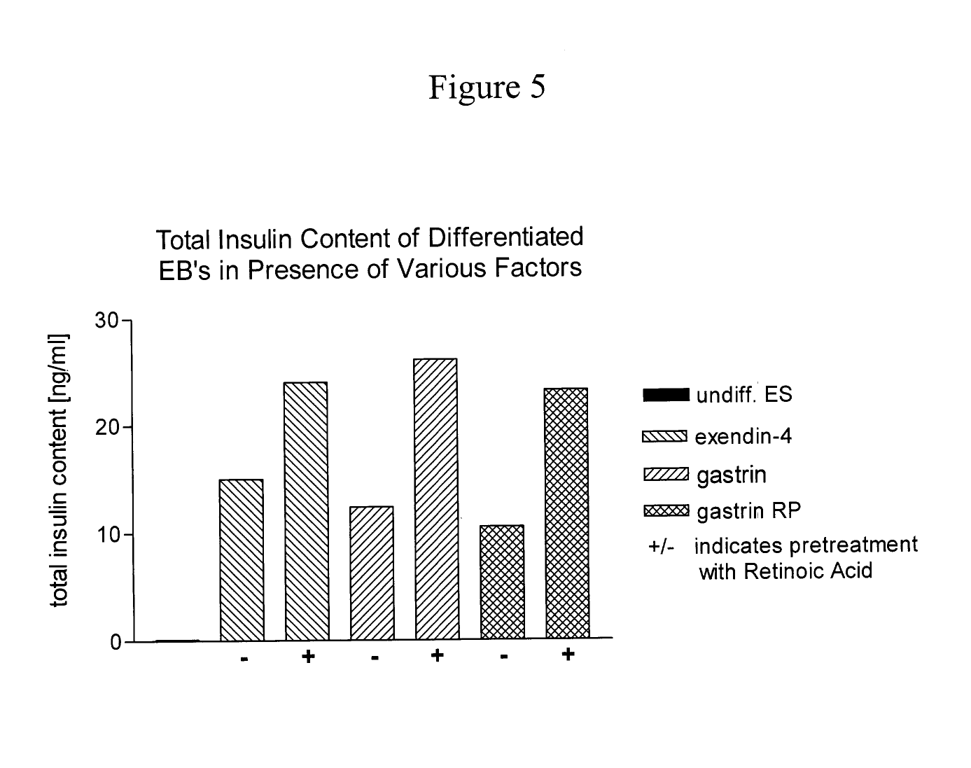

A series of embryoid bodies were prepared as described above in Examples 1-10 (with or without retinoic acid treatment), except that various morphogens (gastrin, gastrin releasing peptide and exendin-4) were added after day 19. The resulting embryoid bodies were collected on day 32 and assayed for insulin content by an insulin specific radioimmunoassay (RIA). For measurement of total insulin content, cell pellets corresponding to 100 EB's per each differentiation condition were washed twice in phosphate buffer solution (PBS), resuspended in 1 ml nanopure water and sonicated. Insulin levels were measured using the Sensitive Rat Insulin RIA Kit (sensitivity 0.02 ng/ml, Linco Research, Inc.) according to the manufacturer's instructions with known calibration standards. The results plotted in FIG. 5 demonstrate that embryoid bodies treated with retinoic acid and morphogen produce much higher levels of insulin that embryoid bodies treated with morphogen alone. These results show that retinoid treatment can be used to augment the differentiation effects of other pancreatic morphogens.

What is claimed is:

1. A method of inducing stem cell differentiation, comprising treating isolated stem cells with a retinoid under conditions effective to cause at least a portion of the stem cells to differentiate into hepaticopancreatic tissue.

2. The method of claim 1 wherein the stem cells are obtained from a stem cell source selected from the group consisting of placenta, bone marrow, adipose tissue, neural tissue, umbilical cord, blastocyst inner cell mass, and germ cells.

3. The method of claim 1 wherein the stem cells are mammalian embryonic stem cells.

4. The method of claim 1 wherein the retinoid is vitamin A.

5. The method of claim 1 wherein the retinoid is selected from the group consisting of retinol, retinal and retinoic acid.

6. The method of claim 1 wherein the retinoid is retinoic acid.

7. The method of claim 1 wherein the hepaticopancreatic tissue is pancreatic tissue.

8. The method of claim 1 wherein the hepaticopancreatic tissue is pancreatic endocrine tissue.

9. The method of claim 8 wherein the hepaticopancreatic tissue comprises insulin-producing cells.

10. The method of claim 9 wherein the insulin-producing cells are glucose-responsive.

11. The method of claim 1 wherein the hepaticopancreatic tissue is liver tissue.

12. The method of claim 1 wherein the conditions are effective to differentiate at least about 1% of the stem cells into hepaticopancreatic tissue.

13. The method of claim 1 wherein the conditions are effective to differentiate at least about 5% of the stem cells into hepaticopancreatic tissue.

14. The method of claim 1 further comprising treating the isolated stem cells with a morphogen.

15. The method of claim 14 wherein the morphogen is selected from the group consisting of a member of the glucagon-like peptide family, a cAMP raising agent, nicotinamide, a transcription factor, a protein growth factor, and mixtures thereof.

16. The method of claim 15 wherein the morphogen is selected from the group consisting of GLP-1, exendin-4, PDX-1, Ngn-3, gastrin, gastrin-releasing peptide, hepatocyte growth factor, betacellulin, and mixtures thereof.

17. A composition comprising the hepaticopancreatic tissue produced by the method of claim 1.

18. The composition of claim 17 wherein the hepaticopancreatic tissue comprises glucose-responsive insulin-producing cells.

19. The composition of claim 17 comprising about 1% or more of the hepaticopancreatic tissue produced by the method of claim 1.

20. The composition of claim 17 comprising about 10% or more of the hepaticopancreatic tissue produced by the method of claim 1.

21. The composition of claim 20 made by purifying the composition of claim 17.

22. A method of treatment comprising identifying a mammal having an extraintestinal gastrointestinal disorder and administering to the mammal a therapeutically effective amount of the composition of claim 17.

23. The method of claim 22 wherein the extraintestinal gastrointestinal disorder is a hepaticopancreatic disorder.

24. The method of claim 23 wherein the hepaticopancreatic disorder is selected from the group consisting of diabetes, pancreatitis, hepatic cirrhosis, hepatitis, cancer and pancreatico-biliary disease.

25. The method as claimed in claim 23 wherein the hepaticopancreatic disorder is diabetes.

26. The method as claimed in claim 25 wherein the mammal is a human.

27. The method as claimed in claim 26 wherein the hepaticopancreatic tissue comprises glucose-responsive insulin-producing cells.

28. A method of treatment comprising identifying a human having diabetes and administering to the human a therapeutically effective amount of the composition of claim 18.
